(19) United States
(12) Patent Application Publication
(10) Pub. No.: US 2023/0174943 A1
(43) Pub. Date: Jun. 8, 2023

(54) CELLULAR COMPOSITION AND USES THEREOF

(71) Applicant: Orthocell Limited, Murdoch (AU)

(72) Inventors: Ming-Hao Zheng, City Beach (AU); Monique Cannon, Murdoch (AU)

(73) Assignee: Orthocell Limited, Murdoch (AU)

(21) Appl. No.: 17/543,491

(22) Filed: Dec. 6, 2021

(51) Int. Cl. C12N 5/077; A61K 35/32

(52) U.S. Cl. CPC C12N 5/066; A61K 35/32; C12N 2509/00

(57) ABSTRACT

The invention pertains to the technical field of a method for isolating and expanding tendon cells in vitro. The invention also relates to cellular compositions comprising such tendon cells and use thereof to repair tendon tissue. In particular, the present invention relates to a cellular composition comprising isolated tendon cells which cells express tenomodulin (TNMD) and scleraxis (Scx).

### TECHNICAL FIELD

The invention pertains to the technical field of a method for isolating and expanding tendon cells in vitro. The invention also relates to cellular compositions comprising such tendon cells and use thereof to repair tendon tissue.

### BACKGROUND

Tendons are a type of regularly arranged dense soft, connective tissue that bridges and attaches muscles to bone. Tendons are comprised of type I collagen (80% by weight) that are arranged in fibrils. Covering the tendon is a thin fascia tissue membrane. Tendons also contain some elastin fibres, a proteoglycan matrix and proteinaceous filler between the connective tissue fibres.

When tendons are damaged due to trauma, accumulated wear or surgical intervention, they typically take longer than, for example, bone to heal. Tendons have poor spontaneous regenerative capabilities and complete regeneration is almost never achieved despite intensive remodeling.

Patients whose tendon(s) have been ruptured experience pain, reduced mobility, reduced lubrication between the articulating and adjacent tissues and a heightened risk of post trauma scarring, adhesions and pain. In some cases, during the healing process following a traumatically injured tendon, the body may deposit an excess amount of fibrous collagen at the site of injury. Peritendonous adhesions are also a contributor to poor outcomes in patients undergoing tendon surgery.

Following tendon repair surgery, fibroblasts from surrounding tissues migrate into the wound during the healing process leading to the formation of scar tissue. Peacock E K. In: Peacock E K (ed) Wound repair. W B Saunders, Philadelphia, 1984; pp 263-331. The formation of adhesions between the tendon and surrounding tissue reduces the ability of the repaired tendon to glide normally. This limits post-operative rehabilitation as a result of a reduction in range of motion and an increase in inflammatory pain.

One method for the repair of tendons that has been used in the past with variable results is tendon cell therapy. These procedures typically use fibroblasts, amniotic stem cells, bone marrow aspirate or other tendon-related cells that have been cultured in vitro for a period of time and then reimplanted, sometimes within scaffold structures; however, there are many pitfalls in the culture of tendon cells that have ultimately lead to a poor clinical outcome.

Thus, there is a continuing need of improved methods and products that would effectively inhibit fibroblast formation, scarring and adhesion formation in treating ruptured and otherwise injured tendons. A product which would effectively inhibit fibroblast formation, scarring and adhesion formation can be useful for treating ruptured and otherwise injured tendons. The present invention describes a product that can alleviate some of the issues described above.

### SUMMARY OF THE INVENTION

The present invention provides a cellular composition comprising tendon derived cells isolated from tendon.

In one embodiment of the present invention, there is provided a cellular composition comprising isolated tendon cells selected in vitro for both tenomodulin (TNMD) and scleraxis (Scx) expression.

In some embodiments, the cellular composition comprises isolated tendon cells that have been selectively expanded in culture, which cells further express one or more marker selected from the group consisting of Col1 A1, TSP-4, TNC, DCN, FN1, BGN, and FMOD or combination thereof and a pharmaceutically acceptable carrier.

The level of expression of the markers is above zero i.e., there must be a detectable level of expression. In one embodiment, the cellular composition comprises expanded isolated tendon cells that express markers at, or above, 100 copies per μg of cDNA as detected by droplet digital PCR. Preferably, the expression level of markers is a minimum of: Scx 5000 copies per μg of cDNA, Col1A1 5000 copies per μg of cDNA, TNMD 1000 copies per μg of cDNA as detected by droplet digital PCR.

In one embodiment of the present invention, there is provided a method for obtaining a cellular composition comprising tendon cells, whereby said composition comprises at least 70% of viable tendon cells, comprising the steps of: obtaining a mammalian tendon sample; obtaining a tendon cell suspension from said tendon by performing at least one enzymatic dissociation step; culturing said cell suspension under appropriate conditions; selecting tendon cells expressing both tenomodulin (TNMD) and Scleraxis (Scx); and expanding the selected tendon cells under appropriate conditions to achieve a therapeutic amount.

Preferably, the tendon cells are derived from a tendon sample or tendon biopsy, but the tendon cell source could also be an isolated tendon. Depending on the nature of the tendon sample, the method preferably includes a separation step, whereby the tendon cells are at least partially separated from the tendon sheath. This separation can occur mechanically only, by use of for instance forceps, scalpel, or tweezers or by use of dissociation agents such as enzymes. In one embodiment, both methods can be combined.

Dissociated tendon cells are preferably cultured in growth medium. The growth medium may comprise conditioned medium or non-conditioned medium. Examples of suitable conditioned medium include Iscove's Modified Dulbecco's Medium (IMDM), Dulbecco's Minimum Essential Medium (DMEM), or aMinimum Essential Medium (MEM) or equivalent medium. Examples of suitable non-conditioned medium include (IMDM), DMEM or aMEM or equivalent medium. The culture medium may comprise serum (e.g. bovine serum, fetal bovine serum, calf bovine serum, horse serum, human serum, or an artificial serum substitute [e.g. 1% bovine serum albumin, 10 μg/ml bovine pancreatic insulin, 200 μg/ml human transferring, 10⁴M β mercaptoethanol, 2 mM L-glutamine and 40 μg/ml LDL (Low Density Lipoproteins)], or it may be serum free.

In an embodiment, the growth medium comprises 20% v/v fetal bovine serum (FBS) in DMEM™ F12 supplemented with ascorbic acid (0.017 mg/mL).

Once tendon cells have reached a desired level of expansion, they are collected to form a cellular composition in a therapeutic amount for preventing or treating tendon injury. In one embodiment, expanded tendon cells are collected by centrifugation and resuspended in a suitable pharmaceutical-acceptable carrier in a therapeutic amount, typically between 1 to 10×10⁶ tendon cells per ml. Any suitable pharmaceutical-acceptable carrier maybe used as long as it is suitable for use in vivo i.e. inert. In one embodiment, the tendon cells are resuspended in assembly medium (10% v/v human serum in Phenol Red Free DMEM™ with ascorbic acid (0.3% v/v; 0.015 mg/mL) and gentamicin (0.1% v/v; 0.05 mg/mL).

### DESCRIPTION OF FIGURES

FIG. 1 shows total gene expression relative to culture conditions. Gene expression data was analysed under different culture conditions: A—minimum handling; B—day 3 half media change; C—with 5% serum supplementation; D—deprivation medium; and E—sub-confluent passage.

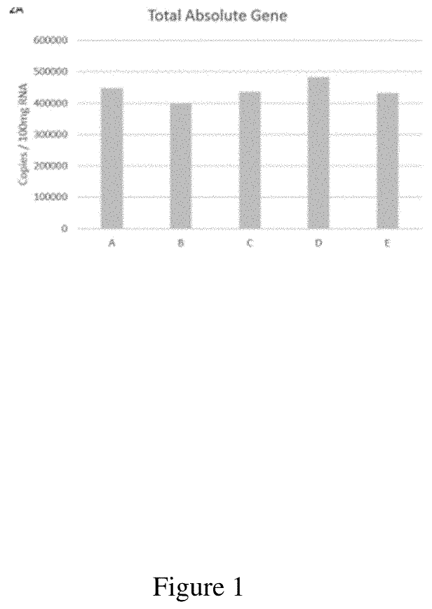

FIG. 2 shows scleraxis (Scx) expression (copies/μg cDNA) distribution plot for PD tendon cells.

FIG. 3 shows collagen I (Col1A1) expression (copies/μg cDNA) distribution plot for PD tendon cells.

FIG. 4 shows tenomodulin (TNMD) expression (copies/μg cDNA) distribution plot for PD tendon cells.

FIG. 5 shows mean ASES score at baseline and at 1, 3, 6 and 12 months after treatment with cellular composition (ATI) or corticosteroid (CS). Error bars=standard error of the mean. Dashed line indicates that there was significant drop-out in the corticosteroid group at month 12 (n=4). The p values are provided for statistically significant differences between groups. NS=no statistically significant difference between groups.

### DETAILED DESCRIPTION OF THE INVENTION

In accordance with the present invention there may be employed conventional molecular biology, microbiology, cell biology, cell culturing and recombinant DNA techniques within the skill of the art. Such techniques are explained fully in the literature. See for example, Sambrook, Fritsch, & Maniatis; DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immnobilized Cells and enzymes IRL Press, (1986); and B. Perbal, A Practical Guide to Molecular Cloning (1984). The invention may also employ standard methods in immunology known in the art.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or able to be arranged with each other to form other additional embodiments or to complement and/or enrich the described embodiment(s), as would be understood by one of ordinary skill in the art.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation.

The expression “% by v/v” (volume percent), here and throughout the description unless otherwise defined, refers to the relative volume of the respective component based on the overall volume of the formulation.

“A”, “an”, and “the” as used herein refers to both singular and plural referents unless the context clearly dictates otherwise. By way of example, “a cell” refers to one or more than one cell.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

By “tissue” is meant a collection of cells having a similar morphology and function.

By “tendon” is meant the dense fibrous connective tissue found in mammals that is made up primarily of collagenous fibres contained within a tendon sheath.

By “tendon sample” is meant a piece, section or biopsy of the tendon, said sample comprising bundles of fibres which contain tendon cells.

The term “tendon cell” as used herein encompasses cells isolated or derived from tendon including cells cultured in vitro.

By “proliferation” is meant an increase in tendon cell number.

By “dissociating a tendon sample” is meant to separate tendon tissue into single tendon cells, smaller cell clusters, or smaller pieces of tissue.

The term “enzymatic dissociation step” is to be understood as any step, involving an enzyme or a solution (digestion solution) comprising an enzyme, which results in complete or partial disconnection of cells normally present within a geometrically arranged two-dimensional or three-dimensional structure, e.g. a tendon sample. As a final result, a tendon cell solution is eventually obtained, preferably mainly consisting of single tendon cells and/or tendon cell clusters. Dissociation occurs preferably by breaking of the intercellular adhesion bonds of tendon cells.

By preference, said enzymatic dissociation step is followed by a mechanical separation of the tendon cells from the tendon sheath. After said mechanical separation of tendon cells from tendon sheath, a cell suspension can be obtained, preferably by means of one or more enzymatic dissociation steps.

In a more preferred embodiment, said enzymatic dissociation step will increase the isolation percentage of the tendon cells. Suitable enzymatic solutions (digestion solution) that can be used for this enzymatic dissociation step may comprise Collagenase type I, Collagenase type II, Collagenase type III, papain and Dispase. Preferably, said enzymatic dissociation step comprises Collagenase type II.

Preferably, the cells will be plated in growth medium (tendon cell culture medium), specifically optimized for culturing and proliferating of tendon cells. In an embodiment of the current invention, the growth medium may comprise conditioned medium or non-conditioned medium. Examples of suitable conditioned medium include IMDM, DMEM, or αMEM or equivalent medium. Examples of suitable non-conditioned medium include Iscove's Modified Delbecco's Medium (IMDM), DMEM or αMEM or equivalent medium. The culture medium may comprise serum (e.g. bovine serum, fetal bovine serum, calf bovine serum, horse serum, human serum, or an artificial serum substitute [e.g. 1% bovine serum albumin, 10 μg/ml bovine pancreatic insulin, 200 μg/ml human transferring, 10⁻⁴M β-mercaptoethanol, 2 mM L-glutamine and 40 μg/ml LDL (Low Density Lipoproteins)], or it may be serum free.

In an embodiment, the growth medium comprises 20% v/v fetal bovine serum (FBS) in DMEM™ F12 supplemented with ascorbic acid (0.017 mg/mL).

In general, the obtained population of tendon cells comprises at least 70% tendon cells. More preferably, the obtained cell population will comprise at least 80% tendon cells, even more preferably at least 90% tendon cells. In a most preferred embodiment, the obtained cell population will consist of 100% pure tendon cells.

In order to ascertain that indeed a pure cellular composition of s has been obtained, the obtained tendon cells are tested by use of suitable markers. In an embodiment of the current invention, the cellular composition comprises tendon cells (s) which express the molecular markers TNMD and Scx as defined herein. Preferably, the tendon cells further express one or more of the markers Col1A1, TSP-4, TSC, DCN, FN1, BGN, and FMOD.

In a most preferred embodiment, a combination of markers is tested in order to come to a conclusive result. The latter allows distinguishing the nature of the isolated and purified tendon cells, and establishing the purity of the obtained sample. Cells which do not comply with the presence of above-mentioned markers should not be considered as tendon cells useful in tendon repair. By the method according to the current invention, a very high yield of pure tendon cell population may be obtained (up to 100%). The latter is of crucial importance to any downstream application.

In some embodiments, the tendon cells of the present invention have minimal expression amounts of the expression markers. In preferred examples, the expression markers are shown as copies per μg cDNA, when measured by droplet digital PCR (ddPCR). ddPCR is used as part of an array of analyses used to evaluate cellular purity, potency and identity (PPI) prior to the use of autologous cells for implantation. ddPCR measures the expression of key phenotype-specific marker genes, with cultured cells expected to maintain expression of marker genes within specified levels in order to be approved for use.

The level of expression of the markers is above zero i.e., there must be a detectable level of expression. In one embodiment, the cellular composition comprises expanded isolated tendon cells that express markers at, or above, 100 copies per μg of cDNA as detected by droplet digital PCR. Preferably, the minimum expression level of markers are as follows: Scx 5000 copies per μg of cDNA, Col1A1 5000 copies per μg of cDNA, TNMD 1000 copies per μg of cDNA, as detected by droplet digital PCR. In some embodiments, the minimum expression of Scx and Col1A1 is 10,000 copies per μg of cDNA

By “cell survival” is meant cell viability.

By “reducing cell death” is meant reducing the propensity or probability that a cell will die. Cell death can be apoptotic, necrotic, or by any other means.

By “cellular factor” is meant any biological agent produced by a cell. While cellular factors isolated from culture media are typically secreted by cells in culture, the scope of the invention is intended to include any factor released from a cultured cell into growth media. In one embodiment, a cellular factor of the invention is secreted by a cell or is released into culture media when a cell breaks open and releases its contents into the growth media. Exemplary cellular factors include HGF, VEGF, SDF-1 alpha, and IGF-1.

By “secreted cellular factor” is meant any biologically active agent that a cell secretes during in vitro culture.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

“Derived from” as used herein refers to the process of obtaining a tendon cell from a subject, tendon sample, or cell culture.

“Detect” refers to identifying the presence, absence or amount of the object to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include any disease or injury that results in a reduction in cell number or biological function, including injury or tissue damage.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “repair” is meant to ameliorate damage or disease in a tissue or organ.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “marker” or “expression marker” is meant any protein or polynucleotide having an alteration in expression level or activity that is associated with culturing tendon cells or tendon-derived cells of the present invention in vitro. The markers of the present invention include scleraxis (Scx), collagen I (Col1A1), tenomodulin (TNMD), thrombospondin-4 (TSP-4), tenascin-C (TNC), decorin (DCN), fibronectin (FN1), biglycan (BGN) and fibromodulin (FMOD). A number of these markers have multiple isoforms and the term marker used herein includes all isoforms.

As used herein, “obtaining” as in “obtaining an agent” includes synthesizing, purchasing, or otherwise acquiring the agent.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

“Patient” refers to an animal preferably a human, to whom treatment, including prophylactic treatment, with the cells, preparations, and compositions of the present invention, is provided. For treatment of those conditions or disease states that are specific for a specific animal such as a human patient, the term refers to that specific animal. A “donor” refers to an individual (animal including a human) who or which donates tendon cells for use in a patient. In most cases the donor will also be the patient i.e. autologous treatment.

“Effective amount” refers to concentrations of components such as growth factors, cells, preparations, or compositions effective for producing an intended result including proliferation of tendon cells, or treating a disease or condition with cells, preparations, and cellular compositions of the invention, or for effecting a transplantation of cells within a patient to be treated.

“Therapeutic amount” refers to the volume or number of expended tendon cells in the cellular composition of the invention that can prevent or treat tendon injury. The volume or number of tendon cells will depend upon the patient and the nature of the injury. One example is in the range of 1×10⁶ cells per ml; however, the exact amount or volume will depend on clinical factors.

The terms “administering” or “administration” refers to the process by which cells, preparations, or compositions of the invention are delivered to a patient for treatment purposes: Cells, preparations, or compositions may be administered a number of ways including parenteral (e.g. intravenous and intraarterial as well as other appropriate parenteral routes), oral subcutaneous, inhalation, or transdermal. Cells, preparations, and compositions of the invention are administered in accordance with good medical practices taking into account the patient's clinical condition, the site and method of administration, dosage, patient age, sex, body weight, and other factors known to physicians.

“Transplanting”, “transplantation”, “grafting” and “graft” are used to describe the process by which cells, preparations, and compositions of the invention are delivered to the site within the patient where the cells are intended to exhibit a favourable effect, such as repairing damage to a patient's tissues, treating a disease, injury or trauma, or genetic damage or environmental insult to an organ or tissue caused by, for example an accident or other activity. Cells, preparations, and compositions may also be delivered in a remote area of the body by any mode of administration relying on cellular migration to the appropriate area in the body to effect transplantation.

“Essentially” refers to a population of cells or a method which is at least 20+%, 30+%, 40+%, 50+%, 60+%, 70+%, 80+%, 85+%, 90+%, or 95+% effective, more preferably at least 98+% effective, most preferably 99+% effective. Therefore, a method that enriches for a given cell population, enriches at least about 20+%, 30+%, 40+%, 50+%, 60+%, 70+%, 80%, 85%, 90%, or 95% of the targeted cell population, most preferably at least about 98% of the cell population, most preferably about 99% of the cell population. In certain embodiments the cells in an enriched tendon or tendon cell population of the invention comprise essentially cells expressing the markers Scx and TNMD and preferably also any one or more of markers Col1A1, TSP-4, TSC, DCN, FN1, BGN, FMOD or combinations thereof.

“Isolated” or “purified” refers to altered “by the hand of man” from the natural state i.e. anything that occurs in nature is defined as isolated when it has been removed from its original environment, or both. In an aspect, a population or composition of cells is substantially free of cells and materials with which it may be associated in nature. By substantially free or substantially purified is meant at least 50% of the population are the target cells, preferably at least 70%, more preferably-at least 80%, and even more preferably at least 90% are free of other cells. Purity of a population or composition of cells can be assessed by appropriate methods that are well known in the art.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

Methodologies

The present invention concerns a method for obtaining a cellular composition comprising tendon cells isolated from mammalian tendon, whereby said composition comprises at least 70% of viable tendon cells. Due to their proliferative nature, these compositions can be used for various tendon repair requirements.

The method according to the current invention can be performed on all types and origins of tendon tissue. Preferably mammalian tendon will be the basis of the obtained cellular composition. Said mammalian tendon can be obtained for instance from, but not limiting to humans, mice, dogs, cats, sheep, cows, pigs, horses, rabbits, rats, guinea pigs, other rodents, etc.

In one of the preferred embodiments, tendon cells are isolated and purified from a human subject to be treated i.e. autologous tendon cells.

Isolation of Tendon Cells

In one embodiment, a tendon sample is obtained by standard biopsy from a subject or patient. The tendon is mechanically and/or chemically dissociated before straining through a sieve or strainer to produce individual tendon cells. The tendon cells are then washed, counted and resuspended in growth media using standard tissue culture techniques.

Media and reagents for tissue culture are well known in the art (see, for example, Pollard, J. W. and Walker, J. M. (1997) Basic Cell Culture Protocols, Second Edition, Humana Press, Totowa, N.J.; Freshney, R. I. (2000) Culture of Animal Cells, Fourth Edition, Wiley-Liss, Hoboken, N.J.). Examples of suitable media for incubating tendon cells include, but are not limited to, Dulbecco's Modified Eagle Medium (DMEM), RPMI media, Hanks' Balanced Salt Solution (HBSS) phosphate buffered saline (PBS) and other media known in the art. Examples of appropriate media for culturing cells of the invention include, but are not limited to, Dulbecco's Modified Eagle Medium (DMEM), RPMI media. The media may be supplemented with fetal calf serum (FCS) or fetal bovine serum (FBS) as well as antibiotics, growth factors, amino acids, inhibitors or the like, which is well within the general knowledge of the skilled artisan.

In some embodiments, the growth medium may comprise conditioned medium or non-conditioned medium. Examples of suitable conditioned medium include IMDM, DMEM, or αMEM or equivalent medium. Examples of suitable non-conditioned medium include Iscove's Modified Delbecco's Medium (IMDM), DMEM or αMEM or equivalent medium. The culture medium may comprise serum (e.g. bovine serum, fetal bovine serum, calf bovine serum, horse serum, human serum, or an artificial serum substitute [e.g. 1% bovine serum albumin, 10 μg/ml bovine pancreatic insulin, 200 μg/ml human transferring, 10⁻⁴M β-mercaptoethanol, 2 mM L-glutamine and 40 μg/ml LDL (Low Density Lipoproteins)], or it may be serum free.

In one embodiment, the growth medium comprises 20% v/v fetal bovine serum (FBS) in DMEM™ F12 supplemented with ascorbic acid (0.017 mg/mL).

Once the tendon cells have been resuspended in growth medium they are cultured for a period of time to expand in number, The proliferation or expansion conditions entail culturing the enriched explant tendon cell preparation for a sufficient period of time so that explant tendon cells in the preparation increase in number while maintaining expression of specific markers. The cells are generally maintained so that the cells complete about 1-100 cell cycles, preferably 5-75 cell cycles; more preferably 2-50, 2-40 or 2-20, most preferably at least about 2-10 or 4-5 cell cycles. This will typically correspond to about 4 to 40 days in culture, preferably about 2-20 days in culture, more preferably at least or about 2-15 days or 4-10 days in culture, and most preferably at least or about 4-8 days in culture.

The frequency of feeding the enriched explant tendon cell preparation is selected to promote the survival and growth of cells with the potential or increased potential to express preferable cellular markers. In an embodiment the cells are fed once or twice a week. The cells may be fed by replacing the entirety of the culture media with new media or preferably with a percentage of media so that cellular factors released by the explant cells are not lost.

In one preferred embodiment, “half media” feeding is used. Without wishing to be bound by theory or hypothesis, the purpose of the half media feeding is to utilize the tendon cell secretome (conditioned media), The term secretome as used in the context of the present invention encompasses proteins shed from the tendon cell surface as well as intracellular proteins released through non-classical secretion pathway or exosomes during in vitro culture. These secreted proteins include numerous enzymes, growth factors, cytokines and hormones or other soluble mediators. It has been reported that cells in culture, other than tendon cells, secrete proteins that may have an effect on cell growth, differentiation, invasion and angiogenesis by regulating cell-to-cell and cell-to-extracellular matrix interactions (see Dowling & Clynes, 2011, Proteomics 2011, 11, 794-804).

Like in most standard cell culture methods, the tendon cells of the present invention will require passaging from time-to-time. In one embodiment of the current invention, passaging involves a trypsinization step. Said trypsinization step is performed under conditions which allow a release of the tendon cells from the tendon structure. Trypsinization can be performed at room temperature (20-25° C.) or at lower or higher temperature (e.g. 37 or 38° C.), for a time period sufficiently long to destroy cell-cell connections.

The methods of the invention may be conducted on a large-scale, for example a cellular composition of the invention may be isolated and/or expanded in a bioreactors.

The method of the present invention leads to a newly created cellular composition comprising a population of explant tendon cells with the potential or increased potential to repair tendons in vivo.

In an aspect of the invention, an isolated and purified cellular composition is provided comprising or comprising essentially of tendon cells characterized by the expression of the markers tenomodulin (TNMD) and scleraxis (Scx).

In some embodiments, the purified cellular composition comprises isolated tendon cells that have been selectively expanded in culture, which cells further express one or more marker selected from the group consisting of Col1A1, TSP-4, TSC, DCN, FN1, BGN, and FMOD or combination thereof.

The level of expression of the markers is above zero i.e., there must be a detectable level of expression. In one embodiment, the cellular composition comprises expanded isolated tendon cells that express markers at, or above, 100 copies per μg of cDNA as detected by droplet digital PCR. Preferably, the expression level of markers are a minimum of: Scx 5,000 copies per μg of cDNA, Col1A1 5,000 copies per μg of cDNA, TNMD 1,000 copies per μg of cDNA as detected by droplet digital PCR. In some embodiments, the minimum expression of Scx and Col1A1 is 10,000 copies per μg of cDNA.

Techniques for ddPCR are well known and understood by those skilled in the art. In one preferred embodiment, ddPCR are performed using the Bio-Rad QX200™ ddPCR system which utilizes microfluidic technology to partition the ddPCR reaction into multiple nanoliter droplet partitions (20,000 water-in-oil emulsion droplets for QX200 system) where individual ddPCR reactions occur. Since the sample is compartmentalized into single droplets (˜1 nL volume), it is considered that an independent PCR reaction takes place in each droplet during amplification.

Amplification occurs over 40 cycles and upon completion, fluorescence is quantified in each sample using a digital reader that establishes amplification thresholds. Poisson statistics are then applied to the ratio of positive droplets to total droplets, ultimately determining the absolute concentration of the target of interest in terms of the number of copies per microliter (cp/μL) in the ddPCR reaction.

All oligonucleotides (primers, probes, and mini-genes) used for ddPCR may be custom-designed and manufactured by, for example, Integrated DNA Technologies (IDT). Upon receipt, fluorescent probes can be diluted to 100 μM with TE buffer (pH 8.0) and stored at −20° C. protected from exposure to ambient light to maintain maximum performance, stability and shelf-life. Primers are typically shipped ‘lab-ready’ in TE buffer (pH 8.0) at a stock concentration of 100 μM and immediately stored at −20° C. upon arrival. Working stocks of all oligonucleotide products are prepared in TE buffer (pH 8.0) at the recommended concentrations for ddPCR (i.e. 5 μM for probes and 25 μM for primers).

ddPCR assay controls are included to assure assay integrity including RNA extraction, cDNA synthesis and ddPCR negative controls to assure absence of extraneous RNA contamination or reagent contamination s as well as excluding self-amplification (via primer dimers etc) as well as no-reverse transcriptase controls and positive controls (minigenes).

Cellular compositions of the invention may also be prepared using positive or negative selection techniques other than the expression markers based on one or more of the characteristics of the cells of the composition as described herein.

Modification of Cells

A cell preparation or cellular composition of the invention may be derived from or comprised of tendon cells that have been genetically modified (transduced or transfected) either in nature or by genetic engineering techniques in vivo or in vitro.

Cells in cell preparations and compositions of the invention can be modified by introducing mutations into genes in the cells (or the cells from which they are obtained) or by introducing transgenes into the cells. Insertion or deletion mutations may be introduced in a cell using standard techniques. A transgene may be introduced into cells via conventional techniques such as calcium phosphate or calcium chloride co-precipitation, DEAE-dextran-mediated transfection, lipofection, electroporation, or microinjection. Suitable methods for transforming and transfecting cells can be found in Sambrook et al., and other laboratory textbooks. By way of example, a transgene may be introduced into cells using an appropriate expression vector including but not limited to cosmids, plasmids, or modified viruses (e.g. replication defective reoviruses, adenoviruses and adeno-associated viruses). Transfection is easily and efficiently obtained using standard methods including culturing the cells on a monolayer of virus-producing cells.

A gene encoding a selectable marker may be integrated into cells of a cell preparation or composition of the invention. For example, a gene which encodes a protein such as (3-galactosidase, chloramphenicol acetyltransferase, firefly luciferase, or a fluorescent protein marker may be integrated into the cells. Examples of fluorescent protein markers are the Green Fluorescent Protein (GFP) from the jellyfish A. victoria, or a variant thereof that retains its fluorescent properties when expressed invertebrate cells.

Another aspect of the present invention relates to genetically engineering the cells in the cell preparations and compositions of the invention in such a manner that they or cells derived therefrom produce, in vitro or in vivo, polypeptides, hormones and proteins not normally produced in the cells in biologically significant amounts, or produced in small amounts but in situations in which regulatory expression would lead to a therapeutic benefit. For example, the cells could be modified such that a protein normally expressed will be expressed at much lower levels. These products would then be secreted into the surrounding media or purified from the cells. The cells formed in this way can serve as continuous short term or long-term production systems of the expressed substance.

Thus, genes can be introduced into cells which are then injected into a recipient where the expression of the gene will have a therapeutic effect.

Other procedures that may be used for selection of tendon cells of interest include, but are not limited to, density gradient centrifugation, flow cytometry, magnetic separation with antibody-coated magnetic beads, affinity chromatography, cytotoxic agents joined to or used in conjunction with a mAb, including, but not limited to, complement and cytotoxins; and panning with antibody attached to a solid matrix or any other convenient technique. The cells can be selected against dead cells, by employing dyes associated with dead cells such as propidium iodide (PI). Preferably, the cells are collected in a medium comprising (alpha MEM), fetal calf serum (FCS), or bovine serum albumin (BSA) or any other suitable, preferably sterile, isotonic medium. Selected cells of the invention may be employed for the isolation of secreted cellular factors as described herein.

In one embodiment, selected cells of the invention comprise a purified population of tendon cells selected for expression of TNMD and Scx. Preferably, the tendon cells further express one or more marker selected from the group consisting of Co/I, TSP-4, TSC, DCN, FN1, BGN, and FMOD or combination thereof. Those skilled in the art can readily determine the percentage of tendon cells in a population using various well-known methods, such as fluorescence activated cell sorting (FACS). Preferable ranges of purity in populations comprising selected cells are about 50 to about 55%, about 55 to about 60%, and about 65 to about 70%. More preferably the purity is at least about 70%, 75%, or 80% pure, more preferably at least about 85%, 90%, or 95% pure. In some embodiments, the population is at least about 95% to about 100% selected cells.

The selected cells may be grown in culture for hours, days, or even weeks during which time their culture medium becomes enriched in one or more secreted cellular factors that support tendon cell proliferation, reduce tendon cell death, preserve tendon function after an injury event, prevent or reduce tendon damage, increase tendon function, increase tendon healing or increase tendon regeneration. Media enriched for such biologically active agents is termed “conditioned media.”

If desired, the tendon cells or their progeny is cultured under conditions that maintain the cells in a proliferative state. In one embodiment, the cells are immortalized to enhance their proliferation. Methods for immortalizing a cell are known in the art and include, but are not limited to, expressing in the cell one or more of dominant negative p53, telomerase, beta catenin, notch and/or a transcription factor or other polypeptide that promotes cell proliferation (e.g., stem cell proliferation). The aforementioned polypeptides may be expressed using any vector suitable for expression in an epicardial progenitor cell (e.g., a viral vector).

Cryopreservation

If required, expanded tendon cells may be cryopreserved for future use using standard techniques.

Uses and Modes of Administration

Once tendon cells have reached a desired level of expansion, they may be collected to form a cellular composition in a therapeutic amount for preventing or treating tendon injury. In one embodiment, expanded tendon cells are collected by centrifugation and resuspended in a suitable pharmaceutical-acceptable carrier in a therapeutic amount, typically between 1 to 10×10⁶ tendon cells per ml. Any suitable pharmaceutical-acceptable carrier maybe used as long as it is suitable for use in vivo i.e. inert. In one embodiment, the tendon cells are resuspended in assembly medium (10% v/v human serum in Phenol Red Free DMEM™ with ascorbic acid (0.3% v/v; 0.015 mg/mL) and a gentamicin (0.1% v/v; 0.05 mg/mL).

The tendon cell preparations and compositions of the invention can be used in a variety of methods (e.g. transplantation) and they have numerous uses in the field of medicine. They may be used for the replacement of tendon, or tendon components or structures which are missing or damaged due to trauma, age, metabolic or toxic injury, disease, idiopathic loss, or any other cause.

Tendons that may be repaired using the cellular compositions of the present invention include tendons with an underlying aetiology of: tendinopathy, tendinitis, tendinosis, tenosynovitis, intrasubstance tendon ruptures, or complete tendon ruptures. Examples of tendon rupture include, but are not limited to, Achilles tendon rupture, patellar tendon rupture, or biceps tendon rupture, which can all be surgically repaired using a method according to an embodiment of the present invention. It is further envisaged that the cellular composition of the present invention could be utilised in the repair of ligaments, which are very similar in architecture to tendons.

Transplantation, grafting or administration, as used herein interchangeably, can include the steps of isolating a cell preparation according to the invention and transferring cells in the preparation into a mammal or a patient. Transplantation can involve transferring the cells into a mammal or a patient by injection of a cell suspension into the mammal or patient, surgical implantation of a cell mass into a tissue of the mammal or patient, or perfusion of a tissue or organ with a cell suspension. The route of transferring the cells may be determined by the requirement for the cells to reside in a particular tissue and by the ability of the cells to find and be retained by the desired target tissue or organ. Where the transplanted cells are to reside in a particular location, they can be surgically placed into a tissue.

The invention may be used for autografting (cells from an individual are used in the same individual), allografting cells (cells from one individual are used in another individual) and xenografting (transplantation from one species to another). Thus, the cells, cell preparations and cellular compositions of the invention may be used in autologous or allogenic transplantation procedures to improve a tendon cell or tendon cell deficit or to repair tissue.

The invention also contemplates a pharmaceutical composition comprising cells, a cell preparation, or cellular composition of the invention, and a pharmaceutically acceptable carrier, excipient, or diluent. The pharmaceutical compositions herein can be prepared by per se known methods for the preparation of pharmaceutically acceptable compositions which can be administered to subjects, such that an effective amount of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the cells, cell preparations, or cellular compositions in association with one or more pharmaceutically acceptable vehicles or diluents, and contained in buffered solutions with a suitable pH and iso-osmotic with the physiological fluids.

Still another aspect of the invention is a kit for producing cellular compositions of the invention comprising cells that have potential or increased potential to form tendon tissue in vivo. The kit includes the reagents for a method of the present invention for producing a cellular composition. This kit preferably would include at least one positive growth factor, and instructions for use.

In one general aspect, the expanded tendon cells are used to seed or populate a construct e.g. scaffold for use in surgical repair of tendon. The construct may be rigid, semi rigid or flexible. In use the construct covers a tendon or part of a tendon and then adheres to the tendon. The construct may be placed over, preferably wrapped around, the tendon after surgical repair of the diseased or damaged tendon during the surgery or clinical procedures. The diseased or damaged tendon can be, for example, tibial tendon, peroneus brevis, longus tendons, etc. The method can further comprise treating the subject with one or more additional therapy for the diseased or damaged tendon, such as physical therapy, NSAIDs, etc.

The invention is further described by the following non-limiting examples which further illustrate the invention, and are not intended to, nor should they be interpreted to, limit the scope of the invention.

### EXAMPLES

### Example 1 Isolation and Purification of Tendon Cells

Biopsy tendon tissue is removed from a patient using sterile forceps and/or sterile scalpel and placed into a sterile petri-dish and diced or minced using a sterile scalpel. Digestion solution (>100U/mL) is added and the dish is placed on an orbital shaker set to operate at 150 rpm for between 2 to 4 hours at 37° C. or until the tendon biopsy material has dissociated into individual cells. The digestion is then stopped by adding growth medium (20% v/v fetal bovine serum (FBS) in DMEM™ F12 supplemented with ascorbic acid (0.017 mg/mL). The tendon cells (or explant tendon cells or explant cells) are then filtered through a 100 μm cell strainer and centrifuged by standard procedure, before being resuspended in 4 ml of growth medium supra and placed in a sterile <25 cm² flask.

The explant cells are then incubated at 37° C. 5% CO₂ for 12-24 hours before visual inspection and media change. Once the explant cells have adhered to the flask they are cultured for 7 days with either a growth medium change after 7 days, a half medium change after about 3 days or a trysinisation by standard procedure depending on the confluency of the cells. The explant cells are seeded to a fairly standard sequence of flasks as they are passaged—usually 25 cm², 75 cm², 2×175 cm² and the like. When the explant cells are established they are usually trypsinised about every 7 days until sufficient explant cells (approximately 10-20×10⁶ cells) have been produced to enable cryopreservation or assemble into a treatment sample. Trypsinisation is typically performed at around 80% confluency.

Human serum is sometimes used in the growth medium supra if cells are not growing well. In that case, supplementation with 5% human serum rather than the standard FBS is used and it is only allowed to sit on the cells for about 3 days. The more common procedure is to use half media change every 3 or so days. The principle is that this practice maintains some of the growth factors generated by the explant cells themselves rather than their removal.

In order to prepare a treatment sample of tendon cells, explant cells that have been expanded (or previously cryopreserved) are trypsinised and washed in phosphate buffered saline (PBS) using standard tissue culture procedures. The expanded tendon cells are then captured by centrifugation and the cell pellet is resuspended to contain 2 to 5×10⁶ cells per mL in pharmaceutically-acceptable carrier medium (sometimes called assembly medium) (10% v/v human serum in Phenol Red Free DMEM™ with ascorbic acid (0.3% v/v; 0.015 mg/mL) and a gentamicin (0.1% v/v; 0.05 mg/mL).

### Example 2 Cellular Markers in Expanded Explant Tendon Cells

Explant tendon cell therapy products produced by the methods of Example 1 may be used to treat injuries of the tendons (chronic degenerative tendinopathy) and articular cartilage, respectively. Droplet digital PCR (ddPCR) is used as part of an array of analyses to evaluate cellular purity, potency and identity (PPI) prior to the use of the expanded tendon cells for implantation. ddPCR measures the expression of key phenotype-specific marker genes, with cultured cells expected to maintain expression of marker genes within specified levels in order to be approved for release.

The ddPCR technique is based on the partitioning of fluorescence-based PCR reaction into thousands of micro-reaction vessels (droplets) of defined volume (˜1 n1) prior to amplification. After amplification, each droplet is analysed through a fluorescence detector (Bio-Rad QX100/QX200 Droplet Reader) and counted as either positive (contains the target) or negative for amplification of the gene of interest. This method allows direct quantitation of the number of target DNA molecules in the reaction, independent of reaction efficiency.

The determination of expression thresholds for cultured tendon cells is determined by evaluating expression levels of genes in primary explant cells following achievement of monolayer culture. As mentioned previously, Applicant has established an expected level of expression based on historical data and a degree of down-regulation is expected in associated with transition from in vivo to in vitro proliferation. It has previously been noted that re-evaluation of gene expression data following the accumulation of sufficient data sets is a necessary part of assay development. Thus, the data will be expressed as reported by the Bio-Rad QuantaSoft software, based on Poisson statistics without the use of calibration curve or standards of known concentration. Thus, this report uses the accumulated data to define minimum copy numbers for gene expression as expected from analysis of the cellular phenotype.

Gene copy numbers (copies/100 μg RNA) were found to be stably expressed under the culture conditions described herein.

The limits for expression were established using a non-parametric approach (percentile value) as this is a non-biased assessment method that is less susceptible to interpretation error. This method is based on published guidance. In this approach, the lower reference limit is established as the value falling at the lower X (2.5, 5, or 10) percentile of the population. For example, if the dataset consists of 109 datapoints, the lower 2.5% reference limit=0.025×109=2.725 (rounded to 3). For this dataset, the 3^{rd} value in the data range (sorted in ascending order of magnitude) therefore represents the lower reference limit or the minimum expression threshold. This therefore establishes the reference range based on the bulk of the analysed population.

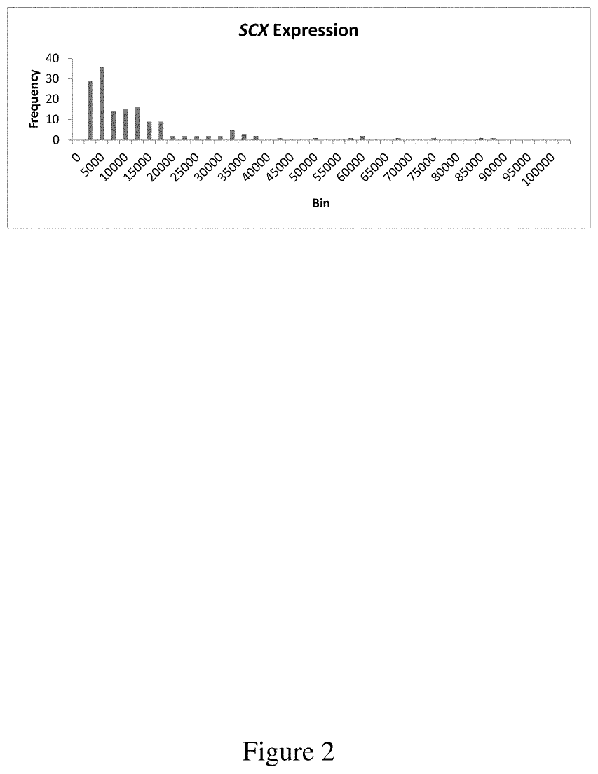

The total amount of cDNA per ddPCR reaction (in final volume of 20 μL) (per Table 1) was used to calculate the copies target gene per μg cDNA. This analysis evaluated all donor cultures processed in order to capture the full range of expression levels expected due to inherent biological variation. Therefore, historical expression data was re-analysed, expressed as copies/μg cDNA and presented as frequency distribution histogram to visualise the results (FIGS. 2-4). The acceptable expression level for all genes, except collagen I, was based upon the observed distribution of data, with the 5^{th} percentile chosen as the cut-off point for acceptability (minimum expression threshold). For collagen 1A1, due to the large distribution in acceptable results, the cut-off point was chosen as the 10^{th} percentile. The data assessed was from samples collected at commencement of in vitro culture (PD) to remove the impact of in vitro culture environment and to more accurately represent the gene expression profile of the parent cell population.

TABLE 1
Sample cDNA concentration data per ddPCR reaction

| | | | Total cDNA per 20 μL |
|---|---|---|---|
| Gene | Vol (μL) | Conc (ng/μL) | ddPCR reaction (ng) |
| COL1A1 | 6.5 | 0.02 | 0.13 |
| SCX | 6.5 | 0.2 | 1.3 |
| TNMD | 11.5 | 6.5 | 75-100 |

In order to establish final thresholds for expression, analysis is dependent upon a minimum of 20 PD samples.

Scleraxis (SCX)

The range of expression of SCX at commencement of in vitro culture is shown in FIG. 2 (n=157). SCX expression ranges from 246-85385 copies/μg cDNA (median=7000 copies/μg cDNA) at PD. With a relatively tight distribution of results, the 5^{th} percentile result is the delimiter for the lower limit of expected expression i.e. the 8^{th} value (0.05×157=7.85) in the data range (sorted in ascending order of magnitude) will be used as the cut-off.

Collagen I (COL1A1)

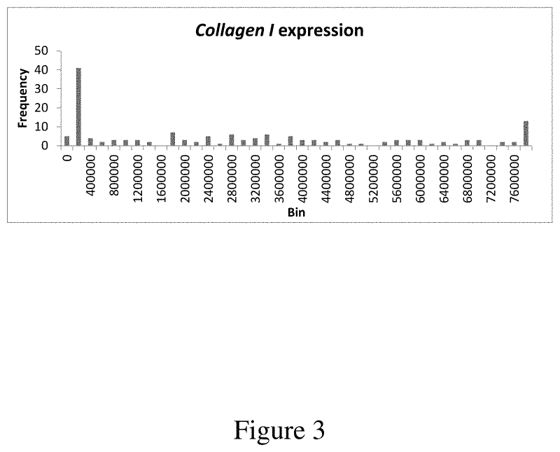

The range of expression of COL1A1 at commencement of in vitro culture is shown in FIG. 3 (n=157). COL1A1I expression ranges from 0-7.69×10⁸ copies/μg cDNA (median=2.26×10⁶ copies/μg cDNA) at PD. With a relatively broad distribution of results, the 10^{th} percentile result is the delimiter for the lower limit of expected expression i.e. the 16^{th} value (0.1×157=15.7) in the data range (sorted in ascending order of magnitude) will be used as the cut-off.

Tenomodulin (TNMD)

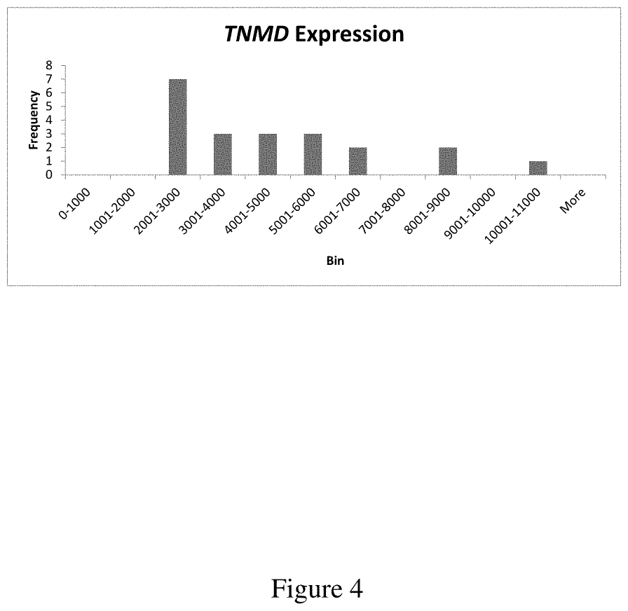

The range of expression of TNMD at commencement of in vitro culture is shown in FIG. 4 (n=21). TNMD expression ranges from 2082-10580 copies/μg cDNA (median=4182 copies/μg cDNA) at PD. With a relatively small distribution of results, the 5^{th} percentile result is the delimiter for the lower limit of expected expression i.e. the 1^{st} value (0.05×21=1.05) in the data range (sorted in ascending order of magnitude) will be used as the cut-off.

Applicant has assessed a large number of cultured samples since implementation of ddPCR. Cells were analysed at commencement of in vitro culture (post-digest/PD samples; timepoint 1) and at the end of culture (prior to assembly for administration—PA samples; timepoint 2). The purpose of assessment at the two timepoints was to assure that expression of specified genes is maintained throughout in vitro cell culture. The stated minimum expression levels must be achieved following culture at the PA time-point. The minimum expression level is based upon historical gene expression data to establish an expectation for the cellular phenotype and therefore assures consistency among manufactured batches.

Expression Limits

The threshold levels for minimum expression based upon the identified percentile value are summarised in Table 2. These values are the minimum expression level required for cultured cells tested prior to assembly of the autologous cellular therapy for the phenotype-specific genes. These data show that Scleraxis may be used as a marker alone (as could Collagen I) or alternatively both markers could be used together to confirm tenocytic phenotype. When the 10^{th} percentile of Collagen I expression is graphed (the lowest 10% of data values), the corresponding Scleraxis expression levels show a tendency for higher expression. As such, observed expression levels are consistent with the expectation that transcription factors targeting a particular gene are higher when the target gene itself is lowly expressed.

TABLE 2
Expression limits (copies/μg
cDNA) for phenotype specific markers

| | SCX | COL1A1 | TNMD |
|---|---|---|---|
| Percentile | 846\hat{ } | 1084* | 2082\hat{ } |
| Threshold | 423 | 542 | 1041 |

\hat{ } = 5^{th} percentile
* = 10^{th} percentile

### Discussion

Analysis of the data with respect to copies/μg cDNA is consistent with the full array of analyses used to determine the cellular characteristics of the cellular products including assessment of morphology and growth characteristics.

Having established that copy numbers is the most appropriate method for evaluation of ddPCR data, Applicant has established minimum expression levels (thresholds) which define the expectation for phenotype-specific gene expression of cultured cells.

### Conclusions

Analysis of ddPCR data expressed as copies/μg cDNA is more relevant than a normalized ratio of gene expression against endogenous or housekeeping gene(s) such as GAPDH as may be reported in the literature. Analysis has shown that the absolute expression of copies/μg cDNA data is more representative of cellular characteristics with respect to PPI than the relative expression based on the normalized ratio.

### Example 3 Randomised, Controlled Study of Tendon Cell Implantation Versus Corticosteroid Injection for Rotator Cuff Tendinopathy and Tear

A cellular composition of the present invention comprising autologous tendon cells expanded in culture by the methods of Example 1 and resuspended in pharmaceutically-acceptable carrier, namely, 10% v/v human serum in Phenol Red Free DMEM™ with ascorbic acid (0.3% v/v; 0.015 mg/mL) and gentamicin (0.1% v/v; 0.05 mg/mL)(also called assembly medium) were characterised by the methods of Example 2 and then used in a minimally invasive treatment to address the underlying pathology of rotator cuff tendinopathy and tear. This randomised, controlled study was designed to investigate the feasibility of the tendon cells of the present invention as an emerging treatment for patients with partial, intrasubstance rotator cuff tear and tendinopathy. Assessments of shoulder function, pain, quality of life, and tendon structure were performed using validated outcome measures before treatment, and for up to 12 months post-treatment.

There were 19 participants in the study who were randomised to and received cellular composition of the present invention, and 11 participants who were randomised to and received corticosteroid treatment. There were no significant differences in baseline characteristics between the groups, and no participants were lost to follow-up during the 12-month post-treatment period. The baseline characteristics confirmed that both participant groups had chronic degenerative rotator cuff pathology and were unlikely to improve without intervention.

This study was conducted to provide preliminary evidence of the efficacy, safety, and tolerability of the cellular composition of the present invention in comparison to corticosteroid injection in participants with chronic degenerative rotator cuff tendinopathy with intrasubstance tendon tear, and to conduct feasibility assessments for a larger, statistically powered efficacy study. The data and analysis presented in this report confirm that the study achieved its goals.

Overall, both study treatments were well tolerated, and no safety concerns relating to the tendon cells of the present invention were identified. All participants in the cellular composition treatment group completed the 12-month follow-up visit, whereas 64% of the corticosteroid group were early withdrawals due to lack of response/treatment failure. Furthermore, 54% of the corticosteroid group received additional treatment for their rotator cuff tendinopathy within 12 months of receiving study treatment.

Participants who received cellular composition treatment had statistically and clinically significantly better post-treatment pain and function scores than participants randomised to receive corticosteroid treatment. Improvements in pain and function scores in the cellular composition implantation group were clinically meaningful, and the majority of participants reported scores that reflected a state of wellbeing. Participants in the corticosteroid group experienced a transient improvement in pain, which peaked at month 3 then returned to baseline, but meaningful improvement in function was not achieved. MRI scan aids in diagnosis of rotator cuff conditions in clinical practice but is not normally used to monitor treatment efficacy due to the lack of widely used and validated quantitative methodologies, and the known phenomenon that tendon abnormalities observed on MRI do not necessarily signal the presence of clinically significant symptoms. Results from analysis of study MRI scans were consistent with this, showing that there was no congruent relationship between tendon structure/tear size and clinical outcomes after treatment.

The results of this study demonstrate that the cellular composition of the present invention is more effective than corticosteroid injection for treatment of rotator cuff tendinopathy with intrasubstance tendon tear. These data provide evidence to support key elements of study design including participant population, sample size calculation, and efficacy outcome measures, for a future pivotal randomised controlled trial.

Briefly, autologous tendon cells were harvested from participants randomised to tendon cell treatment via tendon biopsy under local anaesthetic. A maximum of 3×1 mm strip of tendon was harvested from the superficial surface of the tendon using a 14-gauge biopsy needle. Participants were advised to avoid overuse or excessive repetitive motions for 1-2 days. The patellar tendon was the primary source, although other tendons were selected for biopsy where appropriate.

Approximately 4 weeks post-biopsy, up to 2 ml of autologous human tendon cells (2-5×10⁶ cells/ml) suspended in 10% autologous human serum were injected using an 18-gauge needle. The injection was performed under ultrasound guidance into the tendinopathy/tear site at the affected tendon. Participants were advised to rest for two days and restrict activity to light household/office duties for four weeks.

Participants receiving the corticosteroid treatment received a single, ultrasound guided injection of 1 mL Celestone® Chronodose (betamethasone sodium phosphate 3.9 mg/betamethasone acetate 3 mg) combined with local anaesthetic into the subacromial space.

Efficacy and Safety Variables

Adverse Events

Information on adverse events was collected from visit 1, through to the final study visit. Specific information was solicited from participants at each study visit and via physical examination of the affected limb to capture adverse events associated with study treatment or procedures.

American Shoulder and Elbow Surgeons Shoulder Assessment (ASES)

The ASES was created by the Society of American Shoulder and Elbow Surgeons to provide a standardised method for assessing outcomes in multicentre trials of shoulder and elbow surgery. Patients complete a questionnaire with items pertaining to pain and function, where higher scores indicate worse pain and function. The combined ASES score ranges from 0-100, with a higher score indicating better outcomes. A change in ASES score of 12.0 represents a minimum clinically important difference (MCID). The patient acceptable symptom state (PASS) is reported as 78.6.

Assessment of Quality of Life (AQoL-6D)

The AQol-6D is a validated, health-related, multi-attribute utility quality of life instrument. Patients complete a questionnaire consisting of 20 items spread over 6 dimensions (independent living, relationships, mental health, coping, pain, sensory). The AQoL can be used in cost-utility analysis and calculation of Quality-Adjusted Life Years. It ranges in score from 0 (death) to 100 (perfect health).

Constant Score

The Constant score is the recommended scoring system of the European Society of Shoulder and Elbow Surgery, and is the most commonly used outcome measure used in Europe. It is a validated outcome measure that includes assessments of pain, function, range of motion and abduction strength obtained via patient questionnaire and clinical examination. The Constant score ranges from 0-100 points, with a higher score indicating better outcomes. The MCID for the Constant score has been reported as 10.4, with a PASS of 80.

MRI Scan

3T MRI imaging was performed prior to treatment and at 6 and 12 months post-treatment. If the participant previously received an MRI scan, the pre-treatment MRI was repeated at the discretion of the Investigators. For example, if the scan was conducted greater than 2-3 months prior to participation in the study, the participant received a pre-treatment MRI as their clinical condition could have changed in the intervening time.

Physical Examination

A brief physical examination of the affected limb was performed to document joint abnormalities, function and capture localised adverse events after study treatment.

Simple Shoulder Test

The SST was developed in the US and has been validated in patients with shoulder dysfunction. It measures the functional limitations of the affected shoulder using a 12-item questionnaire completed by the patient. For each item, the patient indicates if they are able or unable to perform an activity, with the score ranging from 0 (worst) to 100 (best). The MCID for the SST has been reported as 16.7, and the PASS reported as 70.

Visual Analogue Scale Pain Assessment (VAS Pain)

The Visual Analogue Scale (VAS) pain score rates pain from 0 (no pain) to 10 (worst pain). Participants will be asked to rate their pain at its worst, at rest, at night, and when performing tasks with the affected arm. An overall pain score for each participant visit was calculated by taking the mean of all ratings provided by the participant at that visit. The MCID and PASS for VAS pain have been reported as 1.4 and 3.0 respectively.

Statistical Analysis

The statistical principles and methods employed in analysis of the study data are described in the Statistical Analysis Plan (SAP, version 1 dated 11 Nov. 2021)

Results

Investigational Time Frame

The investigated started with the first participant randomised on 25 Jul. 2017 (first participant in and ended with the last participant being randomised on 17 Jun. 2020. The final follow-up visit was completed on 18 Aug. 2021 (last participant out).

Participant Disposition

Thirty-five patients signed informed consent and were formally assessed for eligibility. Three did not have suitable partial thickness intrasubstance supraspinatus tendon tears and were excluded. Two patients allocated to the cellular composition group were randomised despite not meeting all eligibility criteria and were subsequently withdrawn from the study prior to receiving treatment. The final analysis set (FAS) consisted of 11 participants who were randomised to and received corticosteroid treatment, and 19 who were randomised to and received cellular composition of the present invention. No participants were lost to follow-up during the 12-month post-treatment period; however, seven participants in the corticosteroid group chose to withdraw and seek further treatment prior to their month 12 visit as permitted in the study protocol.

Demographics and Baseline Characteristics

The mean age of enrolled participants at time of study treatment was 50.5 years (SD 8.5, range 30.3-63.4). There were 10 female and 20 male participants. Mean duration of shoulder symptoms was 23.5 months (SD 18.3. range 7-60), and participants received an average of 4.0 prior treatments before study participation (including corticosteroid injection and physiotherapy).

Analysis of Efficacy

The ASES score comprises patient assessments of pain and function, and ranges from 0 (worst) to 100 (best) points. At baseline, the mean ASES score for participants in the cellular composition group was 74.2 points (SD 15.2). Post-treatment, mean ASES score increased to 82.1 (SD 16.4) at 1 month, 87.1 (SD 13.3) at 3 months, 88.6 (SD 11.4) at 6 months, and 93.3 (SD 7.8) at 12 months. The mean improvement in ASES score at 6 and 12 months (14.4 and 19.1 points, respectively) was greater than the published MCID for the ASES, indicating that on average, participants experienced a meaningful and sustained improvement in pain and function after treatment with the cellular composition. Additionally, at month 6, 72% of participants in the cellular composition group reported an ASES score that was equal to or better than an “acceptable” symptom state (PASS). This increased to 95% of participants at 12 months post treatment.

Participants in the corticosteroid group had a mean ASES score of 62.6 (SD 19.6) at baseline and 63.6 (SD 19.9) at month 1 post treatment. The mean score improved to 74.8 (SD 19.3) at month 3 and decreased slightly at month 6 (mean 74.0, SD 19.8). Seven participants in the corticosteroid group withdrew prior to month 12 due to worsening symptoms or lack of improvement. Four participants completed month 12, with a mean ASES score of 62.9 (SD 23.6). The mean change in ASES score was 11.4 points at month 6, which did not meet the threshold for a meaningful improvement, and there were insufficient data to accurately assess change from baseline at month 12. Only 55% of participants achieved a month 6 ASES score that met or exceeded the acceptable symptom state.

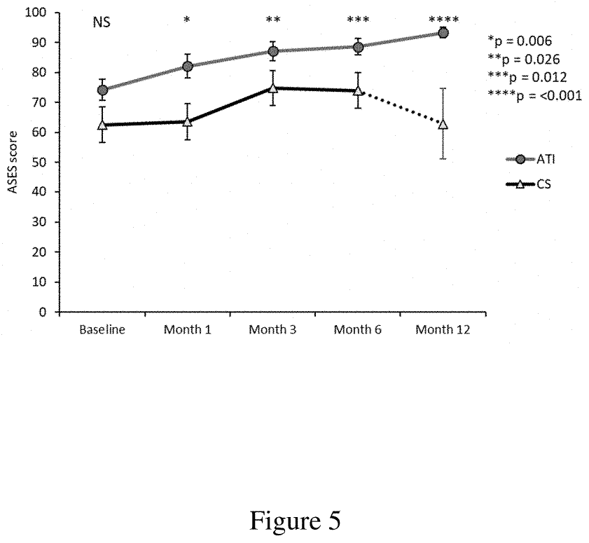

The comparison between groups showed that mean ASES scores were significantly higher in the cellular composition group compared to corticosteroid at all post treatment time points: month 1 (p=0.006), month 3 (p=0.026), month 6 (p=0.012) and month 12 (p=<0.001) (FIG. 5).

Constant Score

The Constant Score consists of patient and physician assessments of pain, function, strength, and range of motion. The final score ranges from 0 (worst) to 100 (best) points. At baseline, the mean Constant score for participants in the cellular composition group was 75.2 (SD 11.6). Post-treatment, mean Constant Score increased to 81.8 (SD 11.5) at 1 month, 83.5 (SD 10.3) at 3 months, 84.9 (SD 10.7) at 6 months, and 86.5 (SD 9.5) at 12 months. The mean improvement in Constant Score at 6 months was 9.7 points; slightly below the published MCID for the Constant Score. At 12 months post treatment, the mean improvement in Constant Score was 11.3 points, indicating that on average, participants experienced a meaningful and sustained improvement in pain and function after treatment with cellular composition. Additionally, at month 6, 73% of participants in the cellular composition group achieved a Constant Score that was equal to or better than an “acceptable” symptom state (PASS). This was maintained at month 12, with 71% of participants achieving a PASS.

Participants in the corticosteroid group had a mean Constant Score of 66.5 (SD 16.3) at baseline. At 1 month post treatment the mean score was stable at 67.6 (SD 18.3), before increasing slightly to 70.6 (SD 19.6) at month 3, and 71.1 (SD 20.5) at month 6. Seven participants in the cortico steroid group withdrew prior to month 12 due to worsening symptoms or lack of improvement. Three participants completed the Constant Score at month 12, with a mean of 65.4 (SD 23.1). The mean change in Constant Score was 4.6 at month 6, which did not meet the threshold for a meaningful improvement, and there were insufficient data to accurately assess change from baseline at month 12. Only 40% of participants in the corticosteroid group achieved a month 6 Constant score that met or exceeded the acceptable symptom state.

The comparison between groups showed that mean post treatment Constant Scores were significantly higher in the cellular composition group compared to the corticosteroid group at month 1 (p=0.020), month 6 (p=0.026), and month 12 (p=0.024).

Simple Shoulder Test (SST)

The SST measures the functional limitations of the affected shoulder based on patient assessments of ability to perform specific tasks with their affected shoulder. Scores range from 0 (worst) to 100 (best) points. At baseline, the mean SST score in the cellular composition treatment group was 67.5 (SD 22.5). Post treatment, SST scores improved to 77.8 (SD 19.0) at month 1, 84.3 (SD 17.8) at month 3, 88.0 (SD 19.0) at month 6, and 90.8 (SD 14.1) at month 12. The mean improvement in SST scores at 6 and 12 months (20.5 and 23.3, respectively), was greater than the published MCID for the SST, indicating that on average, participants experienced a meaningful improvement in pain and function after treatment with Ortho-ATI™. Additionally, at month 6, 89% of participants in the cellular composition group reported an SST score that was equal to or better than an “acceptable” symptom state (PASS). This was similar at 12 months post treatment (84%).

Participants in the corticosteroid group had a mean SST score of 64.4 (SD 22.4) at baseline, which worsened to 60.6 (SD 24.5) at 1 month post treatment, before increasing to 69.7 (SD 22.1) at 3 months, and 75.0 (SD 26.6) at month 6. Seven participants in the corticosteroid group withdrew prior to month 12 due to worsening symptoms or lack of improvement. Four participants completed month 12, with a mean SST score of 77.1 (SD 31.5). The mean change in SST score was 10.6 points at month 6, which did not meet the threshold for a meaningful improvement, and there were insufficient data to accurately assess change from baseline at month 12. At month 6, 72% of participants had an SST score that met or exceeded the acceptable symptom state.

The comparison between groups showed that mean post treatment SST scores were significantly higher in the cellular composition group compared to the corticosteroid group at month 3 (p=0.041) and month 12 (p=0.046).

Visual Analogue Scale Pain Assessment (VAS Pain)

The VAS pain assessment ranges from 0 (no pain) to 10 (worst pain). Participants were asked to rate their pain when at its worst, at rest, lifting a heavy object, performing a repetitive task, and at night. An overall pain score was obtained by taking the mean of reported scores for individual ratings. At baseline, the mean VAS pain score in the cellular composition group was 4.8 (SD 1.8). Post treatment, mean VAS pain scores improved to 2.8 (SD 1.9) at 1 month, 2.3 (SD 2.0) at 3 months, 2.3 (SD 2.1) at 6 months, and 1.6 (SD 1.3) at 12 months. The mean improvement in VAS pain score at 6 and 12 months (−2.5 and −3.2, respectively), was greater than the published MCID for VAS pain, indicating that on average, participants achieved a meaningful reduction in pain after treatment with cellular composition. Additionally, at month 6, 67% of participants in the cellular composition group reported a VAS pain score that was equal to or better than an “acceptable” symptom state (PASS), which improved to 84% of participants at 12 months post treatment.

Participants in the corticosteroid group had a mean VAS pain score of 5.2 (SD 1.6) at baseline, which improved only marginally at one month post treatment (mean 5.1; SD 2.0). The mean score then improved to 2.8 (SD 2.0) at month 3, before worsening to 4.2 (SD 1.9) at 6 months post treatment. The mean improvement in in VAS pain score from baseline at 6 months (−1.0) was below the threshold for meaningful improvement. Additionally, only 27% of participants achieved a VAS pain score that was equal to or better than the published PASS at month 6. Seven participants in the corticosteroid group withdrew prior to month 12 due to worsening symptoms or lack of improvement. Four participants completed month 12, with a mean VAS pain score of 4.3 (SD 2.0).

Mean VAS pain scores improved in both treatment groups at 3 months post treatment. The cellular composition group had a sustained reduction in pain at 6 months, which continued out to 12 months post treatment. In contrast, the reduction in pain in the corticosteroid group was transient, with VAS pain scores increasing between months 3 and 6. The comparison between groups showed that mean VAS pain scores were significantly lower in the cellular composition group compared to the corticosteroid group at all post treatment time points: month 1 (p=0.002), month 3 (p=0.047), month 6 (p=0.010) and month 12 (p=<0.001).

Assessment of Quality of Life (AQoL-6D)

The AQoL-6D score ranges from 0 to 100 (higher scores indicate a better health state). At baseline, the mean AQoL-6D score in the cellular composition group was 78.0 (SD 9.3). Post treatment, mean scores improved to 83.1 (SD 8.4) at 1 month, 83.5 (SD 8.6) at 3 months, 85.2 (SD 7.1) at 6 months, and 87.5 (SD 6.8) at 12 months.

Participants in the corticosteroid group had a mean AQoL-6D score of 74.6 (SD 8.5) at baseline, and scores remained relatively stable at 1 month (72.0; SD 11.9), 3 months (77.2; SD 9.2) and 6 months (79.4; SD 8.7) post treatment. Seven participants in the corticosteroid group withdrew prior to month 12 due to worsening symptoms or lack of improvement. Four participants completed month 12, with a mean AQoL-6D score of 76.6 (SD 19.0).

Both groups showed small improvements in mean AQoL-6D scores post treatment, with the cellular composition group improving by 7.2 points at month 6, compared to 4.8 points in the corticosteroid group. The mean improvement in the cellular composition group at month 12 was 9.5 points. The cellular composition group had a significantly better mean AQoL-6D score at month 1 compared to the corticosteroid group (p=0.010), however there were no statistically significant differences between groups at other time points.

MRI Scan

All participants received MRI assessment at baseline (pre-treatment). However, some post treatment MRI scans were not performed. Six of the eight MRI scans that were not done were due to COVID-19 restrictions. In the corticosteroid group, all participants underwent MRI at 6 months post-treatment. In the cellular composition group, 17 out of 19 participants at 6 months and 16 out of 19 participants at 12 months underwent MRI.

All participants showed rotator cuff pathology at baseline with variation in partial thickness intrasubstance supraspinatus tear and different degrees of tendinopathy. Clinical assessment of MRI showed that patients who received cellular composition trended towards reduction of tear and improvement of tendinopathy, though the outcomes are also variable. The majority of participants who received corticosteroid only had a single post-treatment MRI assessment at 6 months due to high drop-out prior to month 12. MRI assessment showed that most patients who received corticosteroid injection had persistent tendinopathy, with no definitive changes in tendon tear. Semi-quantitative assessment was inconsistent with clinical interpretation of post-treatment MRI scans, indicating that there is a lack of reliable and sensitive methodologies for quantifying treatment effect using MRI.

### Conclusions

Statistical analysis showed that the cellular composition group had significantly better results compared to corticosteroid at all post-treatment time points for the ASES and VAS pain outcome measures. Constant score was significantly better in the cellular composition group at month 1, 6, and 12, and SST score was significantly better in the cellular composition group at months 3 and 12, compared to the corticosteroid group. There were no outcome measures or time points at which the corticosteroid group had significantly better post-treatment outcomes than the cellular composition group.

What is claimed is:

1. A cellular composition comprising isolated tendon cells which cells express tenomodulin (TNMD) and scleraxis (Scx).

2. A cellular composition comprising isolated tendon cells selected in vitro for both tenomodulin (TNMD) and scleraxis (Scx) expression.

3. The cellular composition of claim 1, wherein said tendon cells have been selectively expanded in culture.

4. The cellular composition of claim 3, wherein said tendon cells further express one or more marker selected from the group consisting of Col1A1, TSP-4, TSC, DCN, FN1, BGN, and FMOD or combination thereof.

5. The cellular composition of claim 4, further comprising a pharmaceutically-acceptable carrier.

6. The cellular composition of claim 3, wherein said tendon cells express more than 100 copies of each marker per μg of cDNA as detected by droplet digital PCR.

7. A method for obtaining a cellular composition comprising tendon cells, whereby said composition comprises at least 70% of viable tendon cells, comprising the steps of: obtaining a mammalian tendon sample; obtaining a tendon cell suspension from said tendon by performing at least one enzymatic dissociation step; culturing said cell suspension under appropriate conditions; selecting tendon cells expressing both tenomodulin (TNMD) and scleraxis (Scx); and expanding the selected tendon cells under appropriate conditions to achieve a therapeutic amount.

8. The method of claim 7, wherein the tendon cells are derived from a tendon sample or tendon biopsy.
